(19) United States
(12) Patent Application Publication
(10) Pub. No.: US 2023/0230700 A1
(43) Pub. Date: Jul. 20, 2023

(54) METHOD FOR CLASSIFYING MENTAL STATE AND SERVER FOR CLASSIFYING MENTAL STATE

(71) Applicant: HAII Corp., Seoul (KR)

(72) Inventors: Jaejin Kim, Seongnam-si (KR); Chanhyung Kim, Seoul (KR); Seounguk Ha, Seoul (KR); Hoyoung Kim, Seoul (KR); Hunyeop Jeong, Hanam-si (KR); Jeehyun Han, Seoul (KR); Museok Kang, Seoul (KR); Jinhwan Oh, Seoul (KR); Yunyoung Cho, Seoul (KR); Sangho Jin, Incheon (KR); Jeongsang Yoo, Seoul (KR)

(21) Appl. No.: 18/099,374

(22) Filed: Jan. 20, 2023

(30) Foreign Application Priority Data
Jan. 20, 2022 (KR) 10-2022-0008783

(51) Int. Cl. G16H 50/20; G16H 10/60; G16H 40/67

(52) U.S. Cl. CPC G16H 50/20; G16H 10/60; G16H 40/67

(57) ABSTRACT

A mental state classification method in which a server including a communication unit, a memory, and a processor provides a map to a user is provided. The method comprises: receiving personal information, which include at least one of workplace, age, department, and job group, of a plurality of users from a terminal of each of the plurality of users; storing the received personal information of the plurality of users in the memory; generating a plurality of groups by classifying the plurality of users into administrative districts to which workplaces of the users belong; receiving a signal indicating that the user has selected one of the administrative districts; and in response to the received signal, transmitting, to the terminal, information for displaying an average value of a severity of a mental state of the plurality of users corresponding to the selected district by the terminal.

### REFERENCE TO PENDING PRIOR PATENT APPLICATION

This patent application claims benefit of Korean Patent Application No. 10-2022-0008783, filed Jan. 20, 2022, which patent application is hereby incorporated herein by reference.

### FIELD OF THE INVENTION

The present disclosure relates to a mental state classification method and a mental state classification server, and more specifically, a mental state classification method and a mental state classification server for classifying at least one mental state of a user.

### BACKGROUD OF THE INVENTION

Over the past ten years, a number and cost of treatment for mental disorders has steadily increased, and a lifetime prevalence of seventeen mental disorders is 25.4%, indicating that one in four adults has experienced at least one mental disorder in their lifetime. In addition, according to the same survey, the number of psychiatric treatments related to youth and women has steadily increased over the past five years, which is attributed to the high stress and low socioeconomic level of the corresponding generation. Therefore, in consideration of social cost of mental illness, prevention, early detection, and early treatment are of utmost importance.

Moreover, as COVID-19 epidemic is prolonged, the increase in the number of psychiatric patients is accelerating, and one of the causes, ‘anxiety about a rapid economic recession and a surge in the unemployment rate’, can be considered to have been caused by employment shock after COVID-19 incident. In this regard, according to a study by Forbes, M. K., & Krueger, R. F. (The Great Recession and mental health in the United States. Clinical Psychological Science, 7(5), 900-913.), depression and anxiety increased in many countries during a financial crisis, and rising income inequality due to rising unemployment adversely affects life expectancy and suicide rate.

On the other hand, according to the research paper (J Korean Neuropsychiatr Assoc / Volume 46, No 6 / November, 2007), workers with major depression had more days of absenteeism and more early leave than workers without major depression, and workers with major depression were rated much lower in the evaluation of their job performance. In other words, it was suggested that the overall job performance was greatly deteriorated due to major depression. From this, it can be seen that emotional problems of workers have a great influence on productive capacity of workers.

Therefore, it is necessary to check and manage mental health of workers in order to increase productivity of a company. Moreover, it can be seen that an importance of psychological health is growing in a social situation where communication is difficult due to a recent epidemic of COVID-19 virus.

On the other hand, in the prior art, in order to understand the mental state of the worker, a user wrote an answer to a questionnaire provided by a clinical expert; and the clinical expert directly classified the mental state of the user based on the answer written in the questionnaire.

However, since workers tend not to disclose their current state of mind to others, in reality, it is difficult for the workers to have opportunities to share their current mental state with others and receive console with each other.

As a result, the workers were often left unattended for a long time without taking any action in a state of poor mental condition, which often worsened it. Accordingly, the workers with unhealthy mental states cause a problem of great reduction in their production capacity.

Therefore, there is an urgent need for research and development that can effectively improve mental states of the workers by periodically checking the mental state of workers and providing an opportunity to comfortably share the mental difficulties they are currently experiencing.

### PRIOR DISCLOSURES

(Patent Document 1) Korean Patent Registration No. 10-2111852
(Non-Patent Document 1) Forbes, M. K., & Krueger, R. F. The Great Recession and mental health in the United States. Clinical Psychological Science, 7(5), 900-913.
(Non-Patent Document 2) J Korean Neuropsychiatr Assoc / Volume 46, No 6 / November, 2007.

### SUMMARY OF THE INVENTION

An object of the present disclosure is to provide a new method capable of providing a user with an accurate and highly reliable classification service of a mental state and a communication space where one can share one’s mind by providing a mental state classification method and a mental state classification server.

According to one aspect of the present disclosure, a mental state classification method in which a server including a communication unit, a memory, and a processor provides a map to a user is provided The mental state classification method may comprise: by the communication unit, receiving personal information, which include at least one of workplace, age, department, and job group, of a plurality of users from a terminal of each of the plurality of users; by the processor, storing the received personal information of the plurality of users in the memory; by the processor, generating a plurality of groups by classifying the plurality of users into administrative districts to which workplaces of the users belong, based on the personal information of the plurality of users; by the communication unit, receiving a signal indicating that the user has selected one of the administrative districts; and in response to the received signal, by the processor, transmitting, to the terminal, information for displaying an average value of a severity of a mental state of the plurality of users corresponding to the selected district by the terminal, wherein the administrative districts constitute a map.

According to one embodiment of the present disclosure, the transmitting information for displaying an average value of a severity of a mental state of the plurality of users may compris: by the processor, importing the map stored in the memory; by the processor, displaying the average value of the severity of the mental state of a plurality of users in the district selected by the user on the map; and by the communication unit, transmitting the map on which the average value is displayed to the terminal as information for indicating the average value of the severity of the mental state of the plurality of users.

According to one embodiment of the present disclosure, the transmitting information for displaying an average value of a severity of a mental state of the plurality of users may comprise: matching the district selected by the user and the average value of the severity of the mental state of the plurality of users corresponding to the selected district and transmitting them to the terminal.

According to one embodiment of the present disclosure, the mental state classification method, after the generating a plurality of groups by classifying the plurality of users into administrative districts, may further comprise: by the processor, generating a plurality of subgroups by further classifying the plurality of groups for each job group of the users; by the processor, calculating average values of severities of mental states of users of each of the plurality of subgroups; and by the communication unit, transmitting the average values of the severities of mental states of a group belonging to the job group selected by the user from among the plurality of subgroups to the terminal to display the average values on the map.

According to one embodiment of the present disclosure, the mental state classification method, after the transmitting information for displaying an average value of a severity of a mental state of the plurality of users, may further comprise: by the communication unit, receiving comment contents input by the terminal of the user; by the processor, storing the received comment contents in the memory; by the processor, registering the received comment contents in a comment bulletin board; and by the communication unit, providing the comment bulletin board in which the comment contents are registered to a terminal of each of the plurality of users.

According to one embodiment of the present disclosure, the mental state classification method, after the generating a plurality of groups by classifying the plurality of users into administrative districts to which workplaces of the users belong, may further comprise: by the processor, generating a plurality of age-subgroups by further classifying the plurality of groups by job group of the user and further classifying by age of the user; by the processor, calculating the average value of the severity of the mental state of users of each of the plurality of age-subgroups; by the processor, controlling to display buttons indicating the age of each of the plurality of age-subgroups on the map; by the communication unit, receiving an age selection signal about the age selected by the user’s terminal from the user’s terminal; and in response to the received age selection signal, by the processor, controlling to display on the map average values of severities of mental states of a group belonging to the age selected by the terminal of the user among the plurality of age-subgroups.

According to one embodiment of the present disclosure, the receiving personal information may comprise: receiving a nickname of each of the plurality of users; by the processor, registering the received comment contents in the comment bulletin board, by the communication unit, providing the terminal of the user with the comment bulletin board displaying the user’s nickname as a comment writer, based on the user’s nickname.

According to other aspect of the present disclosure, a mental state classification server that provides a map to a user, may comprise: a communication unit; a memory; and a processor, wherein the communication unit may be configured to: receive personal information, which include at least one of workplace, age, department, and job group, of a plurality of users from a terminal of each of the plurality of users; and receive a signal indicating that the user has selected one of the administrative districts; and wherein the processor may be configured to: store the received personal information of the plurality of users in the memory; generate a plurality of groups by classifying the plurality of users into administrative districts to which workplaces of the users belong, based on the personal information of the plurality of users; calculate average values of severities of the mental state of the users of each of the plurality of groups based on the severity of the mental states of the plurality of users; and in response to the received signal, transmitting, to the terminal, information for displaying the average value of the severity of the mental state of the plurality of users corresponding to the selected district by the terminal.

According to one embodiment of the present disclosure, the processor may be configured to: import a map stored in the memory; and display the average value of the severity of the mental state of a plurality of users in the district selected by the user on the map, and wherein the communication unit may be configured to transmit the map on which the average value is displayed to the terminal as information for indicating the average value of the severity of the mental state of the plurality of users.

According to one embodiment of the present disclosure, the communication unit may be configured to match the district selected by the user and the average value of the severity of the mental state of the plurality of users corresponding to the selected district and transmit them to the terminal.

According to one embodiment of the present disclosure, the processor may be configured to: generate a plurality of subgroups by further classifying the plurality of groups for each job group; and calculate average values of severities of mental states of users of each of the plurality of subgroups, and the communication unit may be configured to transmit the average values of the severities of mental states of groups belonging to the job group selected by the user from among the plurality of subgroups to the terminal to display the average values.

According to one embodiment of the present disclosure, the processor may be configured to: generate a plurality of age-subgroups by further classifying the plurality of groups by job group of the user and further classifying by age of the user; and control to display buttons indicating the age of each of the plurality of age-subgroups on the map, wherein the communication unit may be configured to receive an age selection signal about the age selected by the user’s terminal from the user’s terminal, wherein the processor may be configured to calculate the average value of the severity of the mental state of users of each of the plurality of age-subgroups, and wherein the communication unit may be configured to, in response to the received age selection signal, transmit the average values of the severities of mental states of a group belonging to the age selected by the terminal of the user among the plurality of age-subgroups to the terminal to display the average values.

According to one embodiment of the present disclosure, the communication unit may be configured to receive comment contents input by the terminal of the user, wherein the process may be configured to: store the received comment contents in the memory; and register the received comment contents in a comment bulletin board, and wherein the communication unit may be configured to provide the comment bulletin board in which the comment contents are registered to a terminal of each of the plurality of users.

According to one embodiment of the present disclosure, the processor may be configured to generate a mental state classification result report including a result of classifying all of the plurality of users and the mental state of at least one of groups in which the plurality of users is classified by department, and wherein the communication unit may be configured to transmit the generated mental state classification result report to a terminal of an administrator managing the plurality of users.

### BRIEF DESCRIPTION OF THE DRAWINGS

FIG. 1 is a diagram showing configurations of a mental state classification server and a terminal according to an embodiment of the present disclosure.

FIG. 2 is a diagram illustrating configurations of a terminal to which a mental state classification service is provided through a mental state classification server according to an embodiment of the present disclosure

FIG. 3 is a flowchart illustrating a mental state classification method according to an embodiment of the present disclosure.

FIG. 4 is a diagram showing a state of a terminal provided with information displaying a result of mental state classification of a plurality of office workers on a map from a mental state classification server according to an embodiment of the present disclosure.

FIG. 5 is a diagram illustrating a terminal provided with a map displaying average values of mental states of user groups classified by district from a mental state classification server according to an embodiment of the present disclosure.

FIG. 6 is a diagram showing a state of a terminal provided with a comment bulletin board having a comment writing function from a mental state classification server according to an embodiment of the present disclosure.

FIG. 7 and FIG. 8 are diagrams illustrating a terminal provided with information, from a mental state classification server, for displaying results of classification of mental states of a plurality of office workers classified by administrative district to which workplaces belong, job group, and age on a map, according to an embodiment of the present disclosure

FIG. 9 is a diagram showing states in which a questionnaire is described before proceeding with a questionnaire for classifying a mental state of a computing device according to an embodiment of the present disclosure.

FIG. 10 is a diagram illustrating states of conducting a questionnaire for classifying a mental state of a computing device according to an embodiment of the present disclosure.

FIG. 11 shows classification reference graphs for discriminating a plurality of mental states through heart rate variability data of a mental state classification server according to an embodiment of the present disclosure.

FIG. 12 to FIG. 15. are diagrams illustrating a part of a mental state classification result report provided to an administrator from a mental state classification server according to an embodiment of the present disclosure.

FIG. 16 and FIG. 17 are diagrams illustrating a part of a mental state classification result report provided to an administrator from a mental state classification server according to another embodiment of the present disclosure.

### DETAILED DESCRIPTION OF THE INVENTION

Hereinafter, with reference to the accompanying drawings, the embodiments of the present disclosure will be described in detail so that those of ordinary skill in the art to which the present disclosure pertains can readily implement them. However, the present disclosure may be implemented in several different forms and is not limited to the embodiments described herein.

In order to clearly explain the present disclosure in the drawings, parts irrelevant to the description are omitted, and similar reference numerals are attached to similar parts throughout the specification.

Throughout the specification, when a part “includes” or “comprises” a certain component, it means that other components may be further included: rather than excluding other components, unless otherwise stated.

It is to be understood that the techniques described in the present disclosure are not intended to be limited to specific embodiments, and include various modifications, equivalents, and/or alternatives of the embodiments of the present disclosure

The expression “configured to (or set to)” as used in this disclosure, depending on the context, can be used interchangeably with, for example, “suitable for”, “having the capacity to,” “designed to”, “adapted to”, “made to”, or “capable of”. The term “configured (or configured to)” is not necessarily means only “specifically designed to” hardware. Instead, in some circumstances, the expression “a device configured to” means that the device is “capable of” with other devices or components. For example, the phrases “a processor configured (or configured to perform) A, B, and C,” “a module configured (or configured to perform) A, B, and C”, means a dedicated processor (for example, it may mean an embedded processor) or a generic-purpose processor (e.g., a CPU or an application processor) capable of performing corresponding operations by executing one or more software programs stored in a memory device.

Hereinafter, an embodiment of the present disclosure will be described with reference to the attached drawings However, in the following description, to avoid unnecessarily obscuring the essentials of the present disclosure, detailed descriptions of well-known functions or configurations will be omitted in the following description.

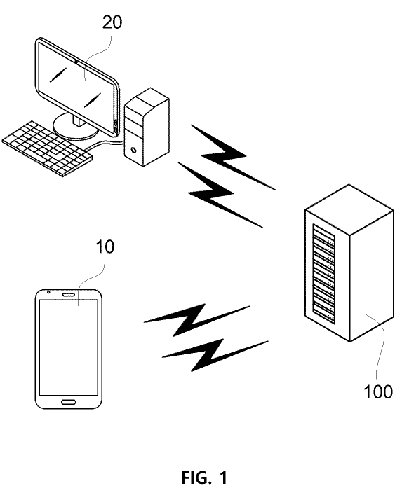

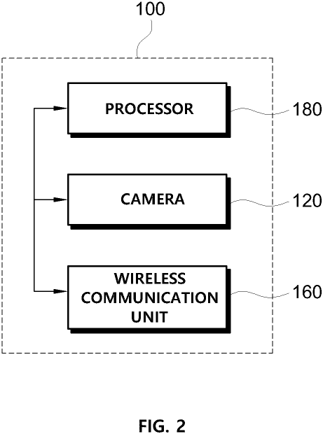

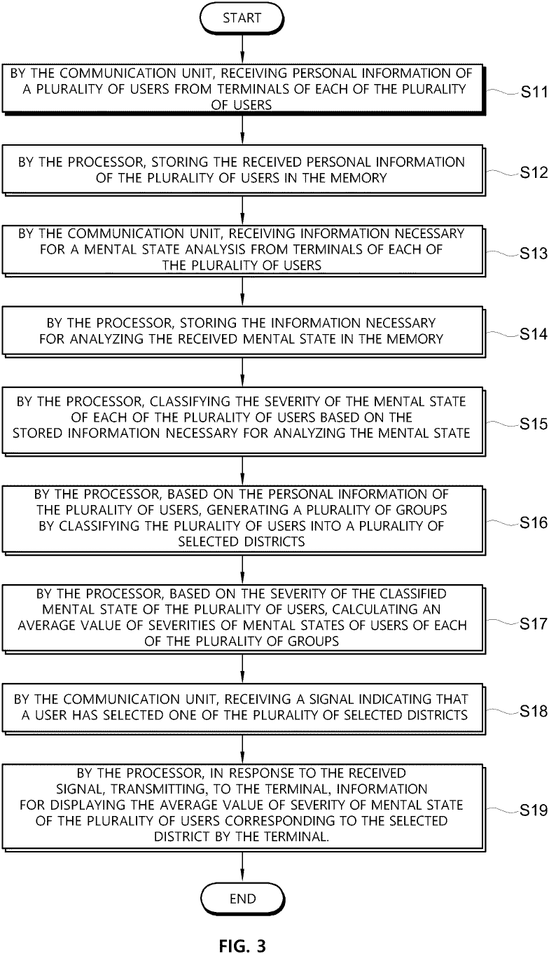

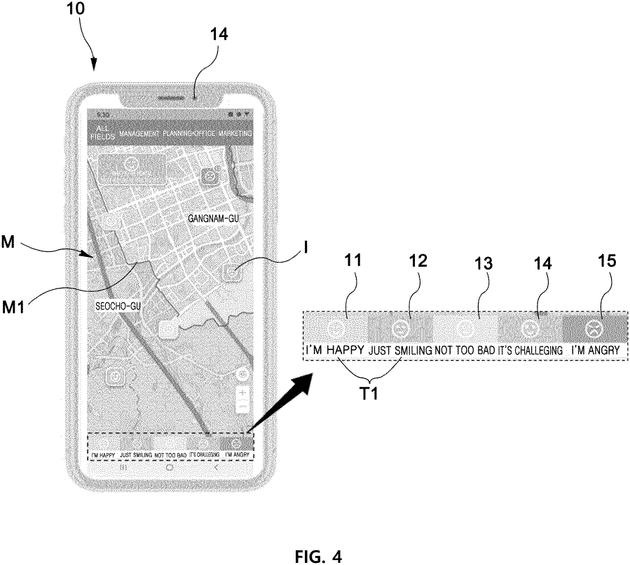

FIG. 1 is a diagram showing the configuration of a mental state classification server 100 and a terminal 10 according to an embodiment of the present disclosure. FIG. 2 is a diagram illustrating configurations of a terminal 10 to which a mental state classification service is provided through the mental state classification server 100 according to an embodiment of the present disclosure. FIG. 3 is a flowchart illustrating a mental state classification method according to an embodiment of the present disclosure FIG. 4 is a diagram showing a state of the terminal 10 provided with information displaying a result of mental state classification of a plurality of users on a map M from the mental state classification server 100 according to an embodiment of the present disclosure

Referring to FIG. 1 to FIG. 4, a mental state classification method according to an embodiment of the present disclosure is a method in which a server 100 equipped with a communication unit 160, a memory 120, and a processor 180 provides a map M to a user, comprising: by the communication unit 160, receiving personal information of the plurality of users from the terminal 10 of each of the plurality of users (S11); by the processor 180, storing the received personal information of the plurality of users in the memory 120 (S12); by the communication unit 160, receiving information necessary for mental state analysis from the terminal 10 of each of a plurality of users (S13); by the processor 180, storing information required for the received mental state analysis in the memory 120 (S14), by the processor 180, classifying a severity of the mental state of each of the plurality of users based on the information necessary for the stored mental state analysis (S15); by the processor, generating a plurality of groups by classifying a plurality of users into a plurality of selected districts M1 based on the personal information of the plurality of users (S16) - the plurality of selected districts M1 constitute the map M -; by the processor, based on the severity of the classified mental state of a plurality of users, calculating an average value of severities of the mental state of each user of a plurality of groups (S17); by the communication unit 160, receiving a signal indicating that the user has selected one of the plurality of selected districts M1 (S18); and by the processor 180, in response to the received signal, transmitting information required for the terminal 10 to display the average value of the severity of the mental state of the plurality of users corresponding to the selected district M1 to the terminal 10 (S19).

In one embodiment, the mental state of each user and the severity corresponding to each mental state may be classified in step S15. For example, a user’s mental state can be classified as at least one of Major Depression Disorder, Anxiety Disorder, Adjustment Disorder, Post Traumatic Stress Disorder (PTSD), Suicidal ideation, and Insomnia Classifications on mental states will be described later. The user, for example, may be classified as having an anxiety disorder and insomnia. In such case, in step S15, the user may be classified by a number or level of severity of anxiety disorder and severity of insomnia. At this time, the severity may be indicated by numbers or may be indicated as high, medium, low, etc. based on a selected range. Severity classification criteria, classification level, grade, etc. can be set by an administrator of the mental classification sever 100.

In one embodiment, the user’s personal information stored in step S12 and the severity obtained in step S15 may be stored in the memory 120 so as to be linked to each other. Accordingly, it is possible to know which level of severity of which mental state the user corresponds to For example, when searching for information, using information that can identify a user (e.g., username, user ID, user nickname, etc.), the processor 180 may store the personal information of the user and the severity obtained in step S15 in the memory 120 so as to be linked to each other, so that the mental state and severity corresponding to the user can be retrieved.

In one embodiment, the processor 180 may be configured to link and store location information and severity of the user’s workplace among the personal information in a memory. For example, the processor 180 may be configured to link and store the user’s workplace information with metadata describing the severity of the mental state Accordingly, the processor 180 may search the severity of the mental state using the user’s workplace location. At this time, the personal information that can determine the user’s identity may not be used as metadata describing the severity of the mental state. That is, when searching or classifying the severity according to location, the processor 180 may store data so that information that can identify the user is not searched. Thus, personal information can be protected.

In one embodiment, the processor 180 may be configured to associate a subgroup and a severity desired to be classified among personal information and store them in a memory. For example, the subgroup may be a group classified by job group J, age G, gender, and the like. For example, the processor 180 may designate and store a subgroup as metadata describing the severity of the mental state. Accordingly, the processor 180 may search the severity of the mental state using detailed information.

In one embodiment, in steps S16 and S17, the processor 180 may classify the severity data stored in the memory into locations, and obtain an average severity value (e.g., a score) according to each location For example, the severity values stored in the memory according to the selected district may be imported, and the average value of these values may be obtained. Accordingly, an average value of severity corresponding to each selected district may be obtained. The selected district may be determined based on an administrative district and may be set by a user or an administrator of the mental state classification server 100. For example, the processor 180 may read the severity values stored in the memory according to the plurality of districts selected in S16 and obtain an average value of these values.

The mental state classification server 100 includes a computing system, hardware running programs, software running on the hardware, and cloud services. The mental state classification server 100 may be connected to other computing devices or other servers through a network In addition, the mental state classification server 100 may include hardware such as a processor 180, a storage or database, and a communication module.

The communication unit 160 may be configured to enable wired/wireless data communication.

The processor 180, as a central processing unit, may be configured to decode and execute instructions comprised of computer language.

The memory 120 may include computer-readable storage media, such as data storage devices that can be accessed by a computing device and provide permanent storage of data and executable instructions (e.g, software applications, programs, functions, etc.). Examples of the memory 120 include volatile and nonvolatile memory, fixed and removable media devices, and any suitable memory device or electronic data storage that holds data for computing device access. The memory 120 may include various implementations of random-access memory (RAM), read-only memory (ROM), flash memory, and other types of storage media in various memory device configurations. Memory 120 may be configured to store executable software instructions (e.g, computer executable instructions) or software applications that may be implemented as modules with processor 180.

Here, “personal information” may be a user’s personal information. For example, the personal information may be at least one of real name, gender, age (date of birth), phone number, workplace information (company name, department, team, job group, position, number of years of service, company location), and nickname.

The terminal 10 includes hardware running a computing system operating program and software running on the hardware and may be connected to each other or to other servers through a network. For example, the terminal 10 may be a smart phone, desktop, laptop, tablet PC, or the like.

The mental state classification method, in the step S11, may additionally obtain a service use agreement from the user. The service use agreement refers to an electronic document for obtaining consent to use personal information of a user in using the mental state classification service provided by the mental state classification method of the present disclosure.

The information required for the analysis of the mental state may be any one or more of a questionnaire for classifying the mental state and an image including the user’s face.

The district M1 may be, for example, an administrative district M1. For example, as shown in FIG. 4, the map M may display administrative districts M1 such as Gangnam-gu and Seocho-gu. In one embodiment, district M1 may be a district selectable by a user. Alternatively, the district M1 may be set based on the location of the user.

As shown in FIG. 4, the average value of the severities of the mental states of the plurality of users may be represented as five images representing stages Individual images of the five images I representing stages may be an image of a wide smile expression I1, an image of a smiling expression I2, an image of an blank expression I3, an image of a hard expression I4, and an image of an angry expression I5. The image I is exemplary, and the image I can be modified in various ways by settings.

In addition, in the mental state classification method of the present disclosure, the average value of the severity of the mental state may be expressed as 5 phrases representing stages T1. The five-level phrase T1 may be expressed by classifying the scale into five levels of “I’m happy”, “I just smile”, “I’m just like that”, “I’m having a hard time”, and “I’m angry” The above phrase T1 is an example, and the phrase T1 can be modified in various ways by settings.

In another embodiment, although not shown in the figure, the mental state classification method of the present disclosure may represent the average value of the severity of the mental state as a percentage or a score.

Therefore, according to this configuration, the mental state classification method of the present disclosure is advantageous in that it can check the mental state of other users in the district M1 to which the user belongs and, especially in a process of identifying which district M1 is bad or stressful, can naturally form empathy among users.

Here, the at least one mental state includes at least one of Major Depression Disorder, Anxiety Disorder, Adjustment Disorder, Post-Traumatic Stress Disorder (PTSD), Suicidal Ideation, and Insomnia Therefore, data for classifying the mental state of the user may be, for example, a user’s answer to a questionnaire related to at least one of Major Depression Disorder, Anxiety Disorder, Adjustment Disorder, PTSD, Suicidal ideation, and Insomnia.

For example, clinical scales of mental states that can be used in the questionnaire are shown in Table 1 below.

TABLE 1

| Category | Name of mental state | Clinical questionnaire tool |
|---|---|---|
| 1 | major depressive disorder | PHQ-9 (Patient Health Questionnaire 9) |
| 2 | anxiety disorder | GAD-7 (Generalized Anxiety Disorder 7) |
| 3 | adjustment disorder | ADNM-4 (Adjustment Disorder-New Module-4) |
| 4 | PTSD | K-PC-PTSD-5 (Korean version of the Primary Care PTSD Screen for DSM-5) |
| 5 | suicidal ideation | P4 (P4 Suicidality Screener) |
| 6 | insomnia | ISI (Insomnia Severity Index) |

According to an embodiment of the mental state classification method, the step of, by the terminal 10, transmitting information required to display the average value of the severity of the mental states of the plurality of users corresponding to the selected district M1 to the terminal 10 may comprise: by the processor 180, importing the map M stored in the memory 120; by the processor 180, displaying the average value of the severity of mental states of a plurality of users in the user-selected district M1 on the map M; and, by the communication unit 160, transmitting the map M on which the average value is displayed to the terminal 10 as information required to indicate the average value of the severity of the mental state of the plurality of users.

According to another embodiment of the mental state classification method, the step of, by the terminal 10, transmitting information required to display the average value of the severity of the mental states of the plurality of users corresponding to the selected district M1 to the terminal 10 may comprise matching and transmitting a district M1 selected by the user and an average value of the severity of mental state of the plurality of users corresponding to the selected district M1. In addition, the mental state classification method, after the step of matching and transmitting the average value of the severity, may comprise, by the terminal 10, displaying a map M stored in a storage device and the average value of the severity of mental state of the plurality of users corresponding to the selected district M1 on the map M

The step of receiving a district selection signal for selecting one or more of the plurality of districts M1 from the terminal 10 of each of a plurality of users may comprise, by a touch pad of the terminal 10, touching and selecting one of the plurality of districts M1.

However, the mental state classification method does not necessarily display only the average value of the severity of mental state of the plurality of users corresponding to the district selected by the user’s terminal 10 on the map M. but the communication unit 160 may transmit average values of the severity to the terminal 10 so that all the average values of the severity of mental state of the plurality of user groups belonging to each of the plurality of districts are displayed on the map M.

Therefore, according to this configuration, the mental state classification method of the present disclosure is advantageous in that a user may obtain a mental comfort, as the user can directly check not only the user’s own district but also the mental states of other users in other districts through the user’s terminal 10, the user can, in a process of checking whether the mental state of various districts is bad or stressful, understand the mental state of other users and naturally form empathy.

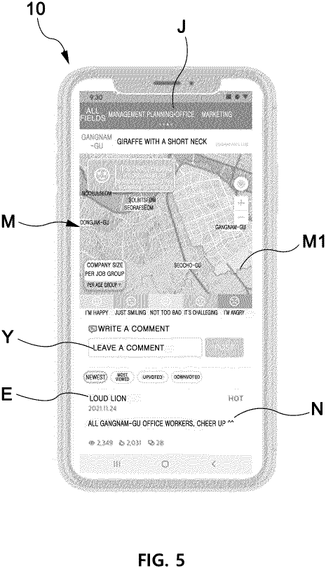

FIG. 5 is a drawing that shows a terminal 10 provided with a map M displaying average values of mental state of user groups classified by district M1 from the mental state classification server 100, according to an embodiment of the present disclosure.

Referring to FIG. 5 in conjunction with FIGS. 1 and 2, after the step of generating a plurality of groups by classifying the plurality of users into a plurality of selected districts M1, the mental state classification method may further comprise: by the processor 180, generating a plurality of subgroups by further classifying the plurality of groups by job group J; calculating an average value of severity of mental state of users of each of the plurality of subgroups; and, by the communication unit 160, transmitting the average value of the severities of mental states of groups belonging to the job group J selected by the user among the plurality of subgroups to the terminal 10 so as to display the average value on a map M.

For example, the job group J may follow the National Competency Standards (NCS) classification.

For example, as shown in FIG. 5, the displaying on a map M the average value of the severities of mental states of groups belonging to the job group J selected by the user among the plurality of subgroups; may further comprise, by the communication unit 160, in response to the selection of the district M1 corresponding to Gangnam-gu on the map M in which the planning/office job group J is selected by the user’s terminal 10, transmitting, to the terminal, the average value of the severity to the terminal so that an image and a phrase displaying the average value of the severity of the mental state of the plurality of users whose workplace is located in Gangnam-gu and belong to the planning/office job group are displayed on the map M.

Therefore, according to this configuration of the mental state classification method of the present disclosure, it is possible for the user to check the mental state of other users of the job group J to which the user belongs. In particular, in a process of checking which mental state is bad or how much stress is, it is possible for the user to understand the mental state of other users in the same job group J, and thus form empathy naturally. Therefore, the method is advantageous that the user can obtain mental comfort from it.

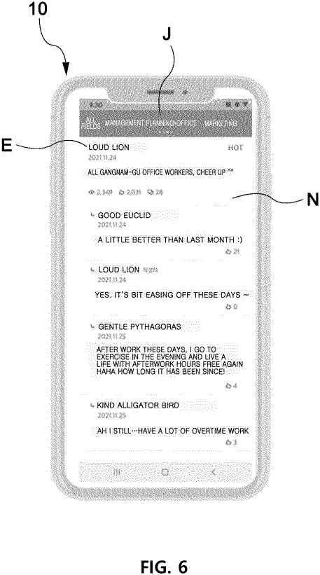

FIG. 6 is a diagram showing a terminal 10 provided with a comment bulletin board having a comment writing function from the mental state classification server 100 according to an embodiment of the present disclosure

Referring to FIG. 6 in conjunction with FIG. 5, the method may further comprise, after the step of displaying the average value of the severity of the mental state of the groups belonging to the selected district M1 in the selected district M1: by the communication unit 160, receiving comment contents inputted by the user’s terminal 10; by the processor 180, storing the received comment contents in the memory 120; by the processor 180, registering the received comment contents on a comment bulletin board: and, by the communication unit 160, providing the comment bulletin board in which the comment contents are registered to the terminal 10 of each of the plurality of users.

The step of receiving the comment contents inputted by the user’s terminal 10 may further comprise, by the communication unit 160, receiving the comment contents inputted in a comment window Y provided in the user’s terminal 10.

That is the comment bulletin board N may include functions of writing comments, displaying registered comments, and displaying registered comments in the order of latest, view counts, highly sympathized, or lowly sympathized.

Therefore, according to this configuration, the mental state classification method of the present disclosure provides opportunity for the users to share their thoughts through the comment bulletin board N and, in a process of exchanging support and comfort between users, it can effectively help users recover their mental state.

Further, the receiving of personal information of the plurality of users from the terminal 10 of each of the plurality of users may comprise receiving a nickname of each of the plurality of users. The step of registering the received comment contents on the comment bulletin board N may comprise, by the communication unit 160, based on the user’s nickname, providing the user’s terminal 10 with the comment bulletin board N on which the user’s nickname is displayed as a comment writer. For example, as shown in FIG. 6, a nickname may be Noisy Lion. Kind Euclid, or Gentle Pythagoras.

Therefore, according to this configuration of the mental state classification method of the present disclosure, a user can be provided with an opportunity to openly talk about one’s mind through an anonymous comment bulletin board N using one’s nickname and recover the user’s mental state in a process of exchanging support and comfort among users.

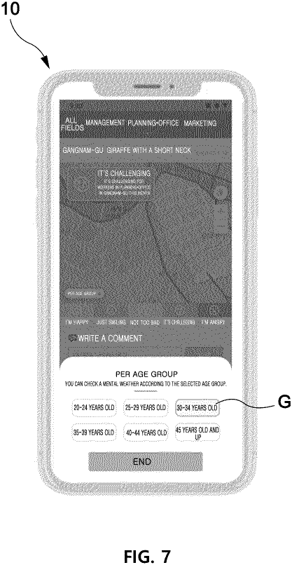

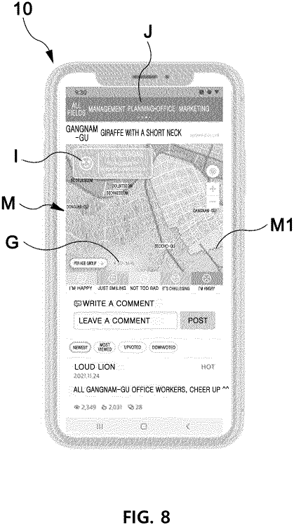

FIG. 7 and FIG. 8 are drawings illustrating appearances of the terminal 10 provided with information for displaying, on the map M, the mental state classification result of a plurality of users classified by the administrative district M1 to which their workplaces belong, job group J, and age G from the mental state classification server 100 according to an embodiment of the present disclosure.

Referring to FIG. 7 and FIG. 8, the mental state classification method according to an embodiment of the present disclosure may further comprise, after the step of generating the plurality of groups by classifying the plurality of users into the plurality of selected districts M1, by the processor 180, generating a plurality of age subgroups by further classifying the plurality of groups by job group J and by age G; by the processor 180, calculating an average value of severity of mental states of the users of each of the plurality of age subgroups; by the processor 180, controlling to display buttons indicating the age of each of the plurality of age subgroups on the map M: by the communication unit 160, receiving, from the user’s terminal 10, an age selection signal related to the age selected by the user’s terminal 10; and by the processor 180, in response to the received age selection signal, controlling to display on the map M the average value of the severity of mental states of the group belonging to an age selected by the terminal 10 of the user among the plurality of age subgroups.

For example, the plurality of users may be classified into 20 to 24 years old, 25 to 29 years old, 30 to 34 years old, 35 to 39 years old, 40 to 44 years old, and 45 years old or older.

For example, as shown in FIG. 7, the mental state classification method of the present disclosure may control the processor 180 to display the average value of the severity of the mental state of a group having a job in the administrative district M1 corresponding to Gangnam-gu, the job group being Planning/Office, and belonging to the age group of thirty to thirty-four years old G on the map M, among the plurality of the users.

Therefore, according to this configuration, the mental state classification method of the present disclosure is advantageous in that a user can obtain mental comfort, as the user can check the mental state of other users of different age groups in the job group J to which the user belongs, and in particular, the user can naturally form empathy in a process of checking the mental state or stress of users in the same age group.

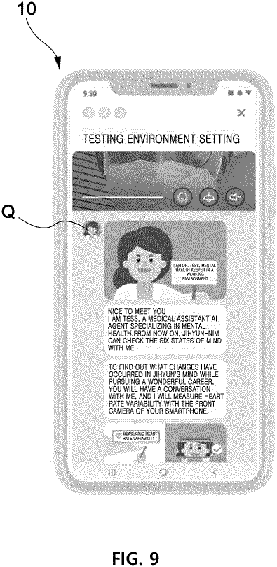

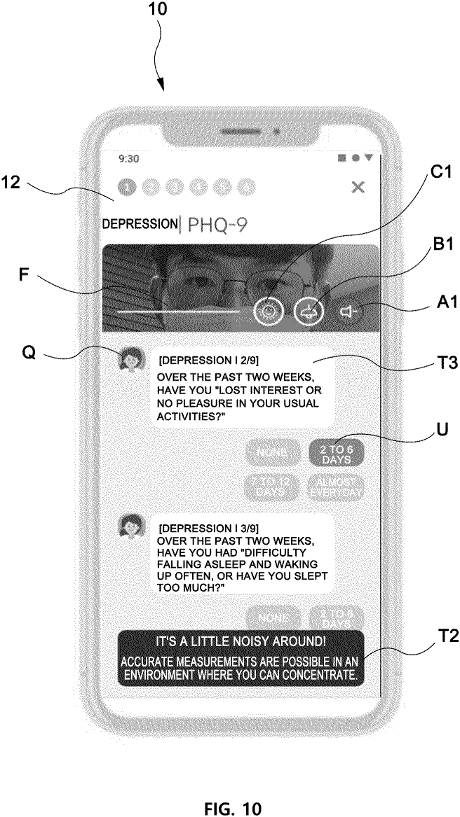

FIG. 9 is a diagram showing an appearance in which a computing device according to an embodiment of the present disclosure explains a questionnaire before proceeding with a questionnaire for mental state classification. FIG. 10 is a diagram illustrating an appearance of conducting a questionnaire for classifying a mental state by a computing device according to an embodiment of the present disclosure

Referring to FIG. 9 and FIG. 10, the step of receiving information necessary for analyzing a mental state from a terminal 10 of each of a plurality of users may comprise by the communication unit 160, providing a questionnaire for classifying the mental state to the terminal 10 of each of the plurality of users; by the communication unit 160, receiving an answer to the questionnaire from the terminal 10 of each of a plurality of users; by the processor 180, controlling the terminal 10 so that a camera 14 of the terminal 10 of each of the plurality of users photographs the user’s face to generate a face image F while conducting a questionnaire for classifying the mental state in each of the terminals 10 of the plurality of users; and by the communication unit, receiving the generated face image F.

In addition, the mental state classification method of the present disclosure may comprise: by the processor 180, checking whether each of the user’s ambient noise, the ambient brightness of the user’s face obtained from the image, and the user’s face position obtained from the image are suitable for a heart rate variability measurement environment; and by the processor 180, based on a result of checking whether the heart rate variability measurement environment is suitable, controlling to display images C1, B1, A1, on the display 12, indicating whether each of the ambient noise, the ambient brightness, and the face position is suitable for the heart rate variability measurement environment.

For example, as shown in FIG. 9, a virtual character may briefly explain to a user about the mental state classification service through a chat message. For example, as in FIG. 10, the step of controlling to display the images C1, B1, and A1 indicating whether the heart rate variability measurement environment is suitable for display on the display 12, in response to the ambient noise not entering a range suitable for the heart rate variability measurement environment, may control, by the processor 180, the display 12 to display the notification phrase T2 “It’s a little noisy around!” as a pop-up window.

Also, as in FIG. 10, the method of providing a questionnaire to the user’s terminal 10 may comprise displaying a question T3 on the chat window of the display 12 by a virtual character Q; and, in response to the questions of the provided questionnaire, selecting a response button U of the display 12 of the user’s terminal 10 and inputting an answer.

Therefore, according to this configuration, the mental state classification method of the present disclosure can take a questionnaire and photograph the user’s face at surrounding noise, ambient brightness, and a position of the camera 14 in an appropriate range for measuring heart rate variability, and thus extract heart rate variability with high accuracy close to the actual heart rate variability of the user.

Here, the heart rate variability (HRV) means a degree of variability of heart rate. That is, the heart rate variability means a minute change between one cardiac cycle and the next cardiac cycle. The heart rate is determined by an influence of the autonomic nervous system on an intrinsic spontaneity of a sinus node, which is related to an interaction between sympathetic and parasympathetic nerves. This interaction changes moment by moment according to changes in the internal/external environment, resulting in a change in heart rate.

In addition, the heart rate variability measurement environment refers to a measurement environment in which a user’s heart rate variability can be extracted through an image F of the user generated in real time by photographing the user using the camera 14. That is, it means an environment in which color changes generated through light reflected in blood vessels under the skin of the user’s face can be clearly distinguished from the images generated through real-time photographing by the camera 14. In addition, the heart rate variability measurement environment may further include an environment in which the user can maintain a mentally stable state.

For example, as shown in FIG. 10, the application program, when providing the questionnaire to the user computing device 10, may be configured so that the virtual character Q delivers the question T3 of the questionnaire to the user in a form of a chat. The application program may be configured so that the user inputs an answer to a question T3 of the questionnaire through a button U

And, the step of classifying the severity of the mental state of each of the plurality of users may comprise: by the processor 180, obtaining a first numerical value indicating a probability corresponding to a mental state of each of the plurality of users based on an answer to the questionnaire received by executing a first algorithm: by the processor 180, extracting heart rate variability data of each of the plurality of users based on the face images of the plurality of users; by the processor 180, obtaining a second numerical value indicating a possibility corresponding to a mental state of each of the plurality of users based on heart rate variability data of the user extracted by executing a second algorithm; and, by the processor 180, executing a third algorithm and obtaining a third numerical value representing a probability corresponding to a mental state of each of the plurality of users based on the first numerical value and the second numerical value.

Therefore, according to this configuration, the mental state classification method of the present disclosure can classify the user’s mental state from each of the user’s answer to the questionnaire for classifying the user’s mental state and the extracted heart rate variability, so that highly reliable mental state classification results can be obtained.

On the other hand, referring back to FIG. 1, the step of extracting the heart rate variability data of the mental state classification method of the present disclosure may comprise, by the mental state classification server 100, extracting heart rate variability (HRV) data by performing real-time image processing on the received image. For example, the method for extracting HRV data comprises by the mental state classification server 100, receiving an image from the terminal 10 in real time and detecting a user’s face in a frame of the received image; defining a measurement area in the detected face; tracking head movement by micro-motion and extracting a color-based micro-motion signal by extracting a minute change in color according to the tracking; converting the extracted facial motion signal into a frequency band through fast Fourier transform (FFT) to extract a power spectrum and normalize it to extract a relative frequency; selecting K of heart rate candidates by comparing a similarity between the relative frequency of the facial motion signal extracted from the image and the established rule base; recognizing an average heart rate of K heart rate candidates extracted from a rule base based on a K-nearest neighbor algorithm through similarity comparison, as a final heart rate; and extracting the HRV variable (HRV data) by calculating it from the final recognized heart rate through a formula of the HRV variable. Examples of the HRV variables are shown in Table 2 below. In addition, in the step of extracting the color-based micro-motion, each micro-motion signal may be normalized and a bandpass filter for a heart rate band may be applied to remove noise other than the heart rate component For example, in the step of defining a measurement region in the detected face, the measurement region may be set to include a middle of a forehead and both cheeks of the user’s face.

For example, the mental state classification method may further comprise providing, to the terminal 10 of the user, a mental state classification report indicating a degree of likelihood that the user’s mental state is Major Depressive Disorder, in terms of images or severity. For example, the mental state may be at least one of Major Depressive Disorder, Anxiety Disorder, Adjustment Disorder, PTSD, Suicidal ideation, and insomnia.

TABLE 2

Descriptions of the HRV variables

| No. | Domain | HRV variable | Explanation |
|---|---|---|---|
| 1 | Time Domain | HR | Average heart rate per minute (bpm) |
| 2 | | SDNN | Standard deviation of intervals between all peaks |
| 3 | | RMSSD | Square root of the mean of the sum of the squares of the differences between adjacent peaks |
| 4 | | pNN50 | Proportion (%) of difference between adjacent peaks greater than 50 |
| | | | msec. |
| 5 | Frequency Domain | VLF | Power values in the 0.0033 to 0.04 Hz band in the frequency domain |
| 6 | | LF | Power values in the 004 to 0.15 Hz band in the frequency domain |
| 7 | | HF | Power values in the 0.15 to 0.4 Hz band in the frequency domain |
| 8 | | VLF (%) | VLF divided by the total power value (power value in the 0.0033 to 0.4 Hz band) |
| 9 | | LF (%) | LF divided by total power value (power value in 0.0033 to 0 4 Hz band) |
| 10 | | HF (%) | HF divided by the total power value (power value in the 0.0033 to 0.4 Hz band) |
| 11 | | InVLF | VLF taken as natural logarithm |
| 12 | | InLF | LF taken as natural logarithm |
| 13 | | InHF | HF taken natural logarithm |
| 14 | | LF,IHF | LF divided by HF |
| 15 | | VLF/HF | VLF divided by HF |
| 16 | | Total Power | Power spectrum band between 0.0033 and 0.4 Hz |
| 17 | | Dominant Power | The power value of the highest peak in the power spectrum |
| 18 | | Dominant Hz | Frequency value (Hz) of the highest peak in the power spectrum |
| 19 | | Peak power | Power spectrum band from -0.015 Hz to +0.015 Hz centered at peak Hz |
| 20 | | Peak Hz | Frequency value (Hz) of the highest peak in the power spectrum band between 0.04 and 0.26 Hz |
| 21 | | Coherence ratio | Peak Power divided by the difference between Total Power and Peak Power |

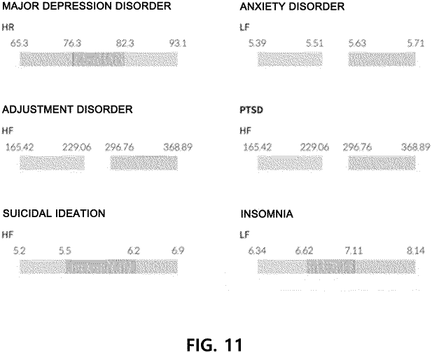

FIG. 11 shows classification reference graphs for discriminating a plurality of mental states through heart rate variability data of a mental state classification server 100 according to an embodiment of the present disclosure.

Referring to FIG. 11, the mental state classification method of the present disclosure, after the step of extracting the heart rate variability data, may perform, by the mental state classification server 100, a step of obtaining a second numerical value representing a probability that the user corresponds to a mental state based on the heart rate variability data of the user extracted by executing the second algorithm. Here, the second numerical value may represent the severity of the user’s mental state. For example, after the mental state classification server 100 extracts HRV variables (HRV data) such as HR value, LF value, and HF value by real-time image processing of the received image, the mental state of the user may be classified by analyzing the extracted HR value, LF value, and HF value as cutoff criteria of a mental disorder screening model. Here, the HR value is related to symptoms of depression, the LF value is related to mental stress and fatigue, and the HF value may decrease when one is suffering from continuous stress, fear, anxiety, or worry.

For example, based on the graph shown in FIG. 11, by the mental state classification server 100. major depressive disorder may be classified as ‘not depressive’ when the HR value was 65.3 to less than 76.3, ‘moderate’ when the HR value was 76.3 to 82.3, and ‘severe’ when the HR value was greater than 82.3 to 93.1.

For example, based on the graph shown in FIG. 11, by the mental state classification server 100, the anxiety disorder may be classified as ‘not anxious’ when the LF value is 5.63 to 5.71, and classified as ‘severe’ when the LF value is 5.39 to 5.51.

For example, based on the graph shown in FIG. 11, by the mental state classification server 100, the adjustment disorder may be classified as ‘no adjustment disorder’ when the HF value is 296.76 to 368.89, and classified as ‘severe’ when the HF value is 165.42 to 229.06.

For example, based on the graph shown in FIG. 11, by the mental state classification server 100, the post-traumatic stress disorder (PTSD) can be classified as ‘not PTSD’ when the HF value is 296.76 to 368.89, and classified as ‘severe’ when the HF value is 165.42 to 229.06.

For example, based on the graph shown in FIG. 11, by the mental state classification server 100, the possibility of suicidal ideation may be classified as ‘not suicidal risk’ when the HF value is less than 6.2 to 6.9, classified as ‘slight’ when the HF value is 5.5 to 6.2, and ‘severe’ when the HF value is less than 5.2 to 5.5.

For example, based on the graph shown in FIG. 11, by the mental state classification server 100, the insomnia may be classified as ‘not insomnia’ when the LF value is greater than 7.11 to 8.14, classified as ‘slight’ when the LF value is 6.62 to 7.11, and classified as ‘severe’ when the LF value is less than 6.34 to 6.62.

Moreover, the mental state classification method of the present disclosure, after obtaining the first numerical value and obtaining the second numerical value, may further include, by the mental state classification server 100, a step of executing a third algorithm and obtaining a third numerical value representing a probability that the user corresponds to a mental state based on the first numerical value and the second numerical value.

Here, the third numerical value may include the severity of the user’s mental state. In one embodiment, the third algorithm may set weights for each of the first and second numerical values and obtain the third values based on the weights. For example, the mental state classification server 100, by executing the third algorithm, may derive the third numerical value representing a final classification result by reflecting the mental state result classified according to the first numerical value by 95% in the final classification result and reflecting the mental state result classified according to the second numerical value by 5% The step of obtaining the third numerical value of the mental state classification method of the present disclosure, by the mental state classification server 100, may further include the step of executing the third algorithm to derive the third numerical value representing the final classification result by multiplying a weight by the second numerical value to the first numerical value.

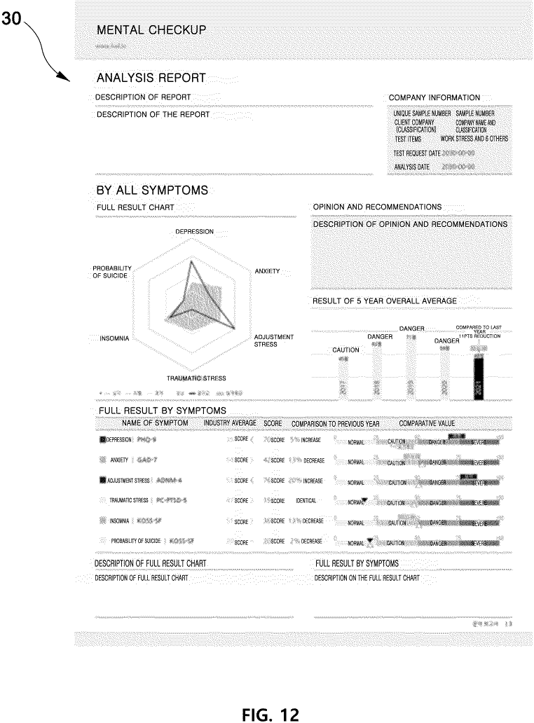

FIG. 12 to FIG. 15 are diagrams illustrating a part of a mental state classification result report 30 provided to an administrator from the mental state classification server 100 according to an embodiment of the present disclosure.

Referring to FIG. 12 to FIG. 15, after the step of classifying the severity of the mental state of each of the plurality of users, the method may further comprise: by the processor 180, generating a mental state classification result report 30 including overall mental state classification results of the plurality of users and mental state classification results of each department of the plurality of users; and by the communication unit 160, providing the generated mental state classification result report 30 to a terminal 20 of an administrator who manages the plurality of users. Details of the mental state classification result report 30 will be described later.

Therefore, according to this configuration, the mental state classification method of the present disclosure may help an administrator who manages a plurality of users recognize a severity of a mental state of the plurality of users and may lead to create a work environment capable of improving the mental state of the plurality of users or provide evidence data leading to prepare a welfare policy Ultimately, it may be possible to effectively enhance abilities of a plurality of users in a job group J.

Meanwhile, referring to FIG. 1 to FIG. 4 again, the present application provides a mental state classification server 100 according to an embodiment of the present disclosure. The mental state classification server 100 may be configured to provide a map M to a user The mental state classification server 100 includes a communication unit 160, a memory 120, and a processor 180.

The communication unit 160 is configured to receive personal information of a plurality of users from a terminal 10 of each of the plurality of users, and to receive information necessary for mental state analysis from the terminal 10 of each of the plurality of users. The communication unit 160 may be configured to receive a signal indicating that one of a plurality of districts has been selected by the terminal of the user.

The processor 180 may be configured: to store the received personal information of the plurality of users in the memory 120: to store information necessary for the received mental state analysis in the memory 120; to classify, based on the stored information necessary for the mental state analysis, the severity of the mental state of each of the plurality of users; to generate, based on the personal information of the plurality of users, a plurality of groups by classifying the plurality of users into a plurality of selected districts M1 that constitute a map M; to calculate, based on the severity of the classified mental state of the plurality of users, an average value of the severity of the mental states of the users of each of the plurality of groups; in response to the received signal, to transmit the average value of the severity of mental state of the plurality of users corresponding to the selected district M1 of the received signal to the terminal 10 and display the average value on the terminal 10; and in response to the received signal, to transmit information necessary to display the average value of the severity of the mental state of the plurality of users corresponding to the selected district M1 to the terminal 10 so the information can be displayed on the terminal 10.

Therefore, according to this configuration, the mental state classification server 100 of the present disclosure is advantageous in that the user can obtain mental comfort, as it is possible for the user to check the mental state of other users in the district M1 to which the user belongs, and the user can naturally form empathy, in particular, in a process of checking which district M1 has a bad mental state or in a lot of stress.

In the mental state classification server 100 according to an embodiment, the processor 180 may read the map M stored in the memory 120 and display the average value of the severity of the mental state of the plurality of users in the district M1 selected by the user on the map M. Thereafter, the communication unit 160 may be configured to transmit the map M on which the average value is displayed to the terminal 10 as information required to indicate the average value of the severity of the mental state of the plurality of users.

In the mental state classification server 100 according to another embodiment, the communication unit 160 may be configured to match and transmit a district M1 selected by the user and an average value of the severity of mental state of the plurality of users corresponding to the selected district M1. In addition, after matching and transmitting the average value of severity, the terminal 10 may display the map M stored in the storage device of the terminal 10 and the average value of the severity of the mental state of the plurality of users corresponding to the selected district M1 on the map M.

Therefore, according to this configuration, the mental state classification server 100 of the present disclosure is advantageous in that a user can obtain mental comfort, as it is possible for the user to directly check mental states of other users in the other district M1 by the user’s own will, thus, in a process of checking whether the mental states of the various districts M1 is bad or is under a lot of stress, the user can understand the mental state of the other users and form empathy naturally

Meanwhile, referring back to FIG. 5 together with FIG. 1 and FIG. 2, the processor 180 can be configured: to generate a plurality of subgroups by further classifying the plurality of groups by job group J; to calculate an average value of the severity of mental state of users of each of the plurality of subgroups; and to display the average value of the severity of the mental states of the subgroup belonging to the job group J selected by the user’s terminal 10 among the plurality of subgroups.

Therefore, according to this configuration, the mental state classification server 100 of the present disclosure is advantageous in that the user can obtain mental comfort as the user can check the mental state of other users of the job group J to which the user belongs and the users can naturally form empathy, in particular, in a process of checking which mental state is bad and how much stress there is, since it is possible to understand the mental state of other users of the same job group J.

Meanwhile, referring to FIG. 6 together with FIG. 5, the communication unit 160 may be configured to receive comment contents inputted by a user. The processor 180 may store the received comment contents in the memory 120 and register the received comment contents on a comment bulletin board N. The communication unit 160 may be configured to provide the terminal 10 of each of a plurality of users with a comment bulletin board N on which the comment contents are registered

Therefore, according to this configuration, the mental state classification server 100 of the present disclosure can provide an opportunity to tell the user’s own story through the comment bulletin board N and, in a process of exchanging support or consolation with other users, it can help users recover their mental state.

In addition, the communication unit 160 may receive a nickname E of each of the plurality of users when receiving personal information of the plurality of users from the terminal 10 of each of the plurality of users. After the processor 180 registers the received comment contents on the comment bulletin board N. based on the user’s nickname, the communication unit 160 may provide the user’s terminal 10 with the comment bulletin board N displaying the user’s nickname E as a writer of the comment

Therefore, according to this configuration, the mental state classification server 100 of the present disclosure can provides an opportunity to openly talk about the user’s own story through an anonymous comment bulletin board N using his or her nickname E and, in a process of exchanging support or consolation between users, it can help users recover their mental state.

On the other hand, referring to FIGS. 7 and 8 again, the processor 180 can generate a plurality of age subgroups by further classifying the plurality of groups by job group J and further classifying it by age and can control so that input buttons marked with ages of each of the plurality of age subgroups may be displayed on the map M

The communication unit 160 may be configured to receive, from the terminal 10 of the user, an age selection signal related to the age G selected by the terminal 10 of the user. The processor 180 may calculate an average value of the severity of mental state of users of each of the plurality of age subgroups and in response to the received age selection signal, may control to display an average value of the severity of mental state of a subgroup belonging to an age G selected by the terminal 10 of the user among the plurality of age subgroups as an image I.

Therefore, according to this configuration, the mental state classification server 100 of the present disclosure has an advantage of obtaining mental comfort for the user, as it is possible for the user to check the mental state of other users of various age subgroups in the job group J to which the user belongs and users can naturally form empathy, in particular, in a process of checking the mental state or stress of users of the same age subgroup.

Meanwhile, referring to FIG. 9 and FIG. 10, the communication unit 160 may receive information necessary for mental state analysis from the terminal 10 of each of a plurality of users. The communication unit 160 may provide a questionnaire for classifying a mental state to the terminal 10 of each of the plurality of users. The communication unit 160 may be configured to receive an answer to the questionnaire from the terminal 10 of each of the plurality of users.

The processor 180 may be configured to control the terminal 10 so that the camera 14 of the terminal 10 of each of the plurality of users photographs the user’s face to generate a face image F while each of the terminals 10 of the plurality of users is conducting a questionnaire for classifying the mental state The communication unit 160 may be configured to receive the generated face image F.

The processor 180 may be configured to obtain a first numerical value indicating a probability corresponding to the mental state of each of the plurality of users based on the received answer to the questionnaire by executing a first algorithm. The processor 180 may be configured to extract heart rate variability data of each of the plurality of users based on the face images F of the plurality of users. The processor 180 may be configured to obtain a second numerical value representing a probability corresponding to the mental state of each of the plurality of users based on the heart rate variability data of the user extracted by executing the second algorithm. The processor 180 may be configured to execute a third algorithm and obtain a third numerical value indicating a probability corresponding to the mental state of each of the plurality of users based on the first numerical value and the second numerical value.

Therefore, according to this configuration, the mental state classification method of the present disclosure has an advantage of obtaining a highly reliable mental state classification result through a questionnaire for classifying a user’s mental state and a highly accurate heart rate variability close to the user’s actual heart rate variability.

FIG. 12 to FIG. 15 are diagrams illustrating a part of a mental state classification result report 30 provided to an administrator from the mental state classification server 100 according to an embodiment of the present disclosure.

Referring to FIG. 12 to FIG. 15, the processor 180 can be configured to generate a mental state classification result report 30 including overall mental state classification results of the plurality of users and mental state classification results of each department of the plurality of users. The communication unit 160 may be configured to transmit the generated mental state classification result report 30 to the terminal 20 of an administrator managing the plurality of users

For example, as shown in FIG. 12, the mental state classification result report 30 may include a result of classifying mental states, such as depression, anxiety, adaptive stress, trauma stress, insomnia, and probability of suicide, of the plurality of users.

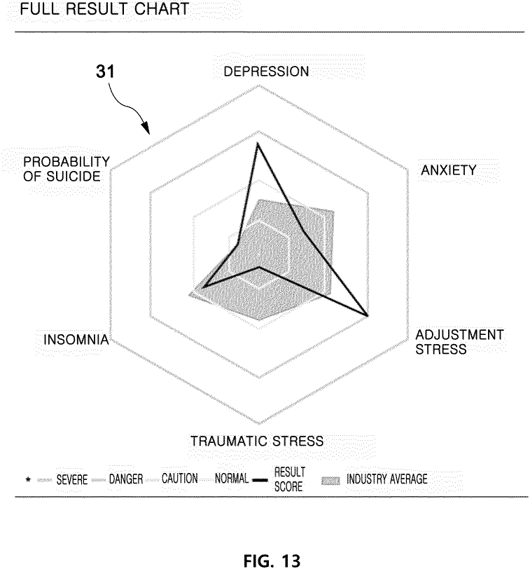

For example, as shown in FIG. 13, the mental state classification result report 30 may include a graph 31 showing the severity of each of depression, anxiety, adaptive stress, trauma stress, insomnia, and probability of suicide of the plurality of users.

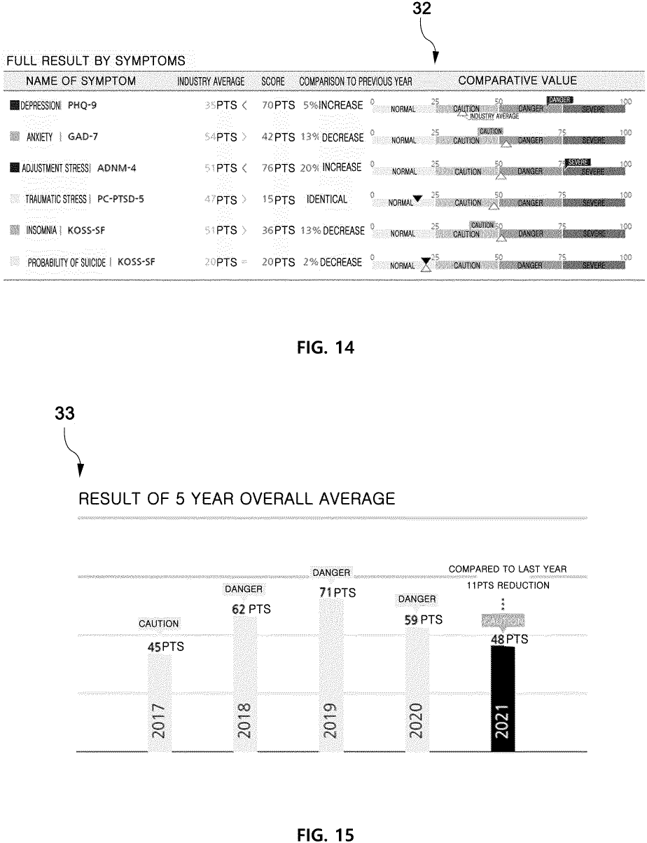

For example, as shown in FIG. 14, the mental state classification result report 30 may include a graph 32 showing industry average values of the severities of mental classification results, severities of mental classification results of the plurality of users of a company, and a comparison value of the severities of the plurality of users of the company compared to last year.

For example, as shown in FIG. 15, the mental state classification result report 30 may include a graph 33 showing data contents of mental classification results of employees for the past five years who worked for a company to which a plurality of users belongs.

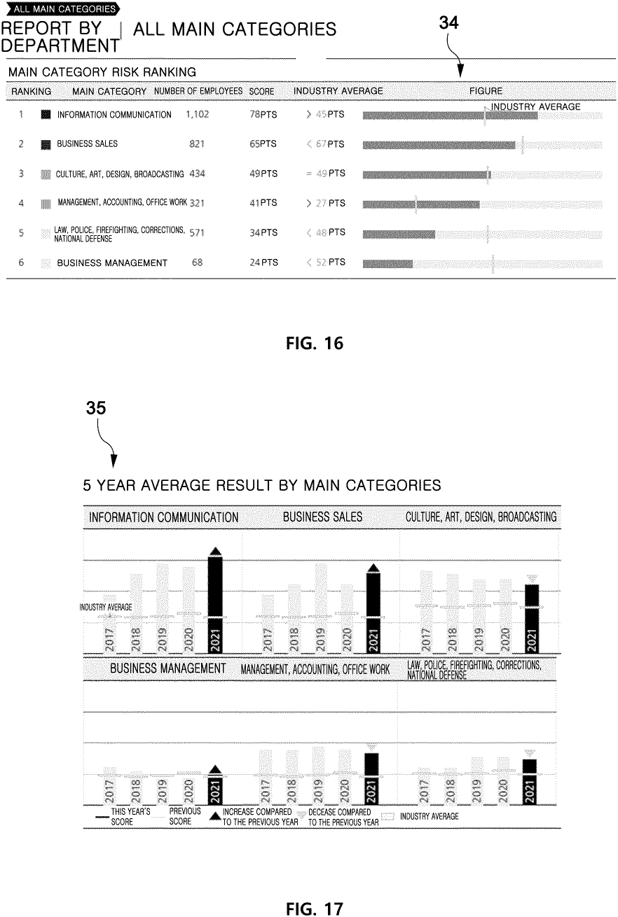

FIG. 16 and FIG. 17 are diagrams illustrating a part of a mental state classification result report 30 provided to an administrator from the mental state classification server 100 according to another embodiment of the present disclosure.

First, referring to FIG. 16, the mental state classification result report 30 includes mental classification result data of a plurality of user groups classified by job group J and a graph 34 showing severities by job group of mental classification results of the plurality of users and industry average values of the severities of mental classification results.

For example, as shown in FIG. 17, the mental state classification result report 30 may include a graph 35 showing data contents of results of mental classification of employees classified by job group J for the past five years for a company to which a plurality of users belongs.

Therefore, according to this configuration, the mental state classification method of the present disclosure can ultimately effectively enhance job ability of a plurality of users, as it helps an administrator who manages a plurality of users recognize the severity of the mental state of the plurality of users and makes it possible to create a work environment that can improve the mental state of the plurality of users or provide evidence data that can lead to prepare a welfare policy.

In the embodiments disclosed herein, the arrangement of the illustrated components may vary depending on the environment or requirements in which the invention is implemented For example, some components may be omitted or some components may be integrated and implemented as one.

In addition, the arrangement order and connection order of some components may be changed.

Although the above has shown and described various embodiments of the present disclosure, the present disclosure is not limited to the specific embodiments described above The above-described embodiments can be variously modified and implemented by those skilled in the art to which the present invention pertains without departing from the gist of the present disclosure claimed in the appended claims and these modified embodiments should not be understood separately from the technical spirit or scope of the present disclosure. Therefore, the technical scope of the present disclosure should be defined only by the appended claims.

The described embodiments of the present disclosure also allow certain tasks to be performed on a distributing computing environment performed by remote processing devices that are linked through a communications network. In the distributed computing environment, program modules may be located in both local and remote memory storage devices.

In the embodiments disclosed herein, the arrangement of the illustrated components may vary depending on the environment or requirements in which the invention is implemented. For example, some components may be omitted or some components may be integrated and implemented as one.

What is claimed is:

1. A mental state classification method in which a server including a communication unit, a memory, and a processor provides a map to a user, comprising:
by the communication unit, receiving personal information, which include at least one of workplace, age, department, and job group, of a plurality of users from a terminal of each of the plurality of users;
by the processor, storing the received personal information of the plurality of users in the memory;
by the processor, generating a plurality of groups by classifying the plurality of users into administrative districts to which workplaces of the users belong, based on the personal information of the plurality of users;
by the communication unit, receiving a signal indicating that the user has selected one of the administrative districts; and
in response to the received signal, by the processor, transmitting, to the terminal, information for displaying an average value of a severity of a mental state of the plurality of users corresponding to the selected district by the terminal,
wherein the administrative districts constitute a map.

2. The mental state classification method of claim 1, wherein the transmitting information for displaying an average value of a severity of a mental state of the plurality of users comprising:
by the processor, importing the map stored in the memory;
by the processor, displaying the average value of the severity of the mental state of a plurality of users in the district selected by the user on the map; and
by the communication unit, transmitting the map on which the average value is displayed to the terminal as information for indicating the average value of the severity of the mental state of the plurality of users.

3. The mental state classification method of claim 1, wherein the transmitting information for displaying an average value of a severity of a mental state of the plurality of users comprising:
matching the district selected by the user and the average value of the severity of the mental state of the plurality of users corresponding to the selected district and transmitting them to the terminal.

4. The mental state classification method of claim 1, after the generating a plurality of groups by classifying the plurality of users into administrative districts, further comprising:
by the processor, generating a plurality of subgroups by further classifying the plurality of groups for each job group of the users;
by the processor, calculating average values of severities of mental states of users of each of the plurality of subgroups; and
by the communication unit, transmitting the average values of the severities of mental states of a group belonging to the job group selected by the user from among the plurality of subgroups to the terminal to display the average values on the map.

5. The mental state classification method of claim 1, after the transmitting information for displaying an average value of a severity of a mental state of the plurality of users, further comprising:
by the communication unit, receiving comment contents input by the terminal of the user;
by the processor, storing the received comment contents in the memory;
by the processor, registering the received comment contents in a comment bulletin board; and
by the communication unit, providing the comment bulletin board in which the comment contents are registered to a terminal of each of the plurality of users.

6. The mental state classification method of claim 1, after the generating a plurality of groups by classifying the plurality of users into administrative districts to which workplaces of the users belong, further comprising:
by the processor, generating a plurality of age-subgroups by further classifying the plurality of groups by job group of the user and further classifying by age of the user;
by the processor, calculating the average value of the severity of the mental state of users of each of the plurality of age-subgroups;
by the processor, controlling to display buttons indicating the age of each of the plurality of age-subgroups on the map;
by the communication unit, receiving an age selection signal about the age selected by the user’s terminal from the user’s terminal; and
in response to the received age selection signal, by the processor, controlling to display on the map average values of severities of mental states of a group belonging to the age selected by the terminal of the user among the plurality of age-subgroups.

7. The mental state classification method of claim 5, the receiving personal information comprising:
receiving a nickname of each of the plurality of users;
by the processor, registering the received comment contents in the comment bulletin board,
by the communication unit, providing the terminal of the user with the comment bulletin board displaying the user’s nickname as a comment writer, based on the user’s nickname.

8. A mental state classification server that provides a map to a user, comprising:
a communication unit; a memory; and a processor,
wherein the communication unit is configured to: receive personal information, which include at least one of workplace, age, department, and job group, of a plurality of users from a terminal of each of the plurality of users; and receive a signal indicating that the user has selected one of the administrative districts; and
wherein the processor is configured to: store the received personal information of the plurality of users in the memory; generate a plurality of groups by classifying the plurality of users into administrative districts to which workplaces of the users belong, based on the personal information of the plurality of users; calculate average values of severities of the mental state of the users of each of the plurality of groups based on the severity of the mental states of the plurality of users; and in response to the received signal, transmitting, to the terminal, information for displaying the average value of the severity of the mental state of the plurality of users corresponding to the selected district by the terminal.

9. The mental state classification server of claim 8,
wherein the processor is configured to: import a map stored in the memory; and display the average value of the severity of the mental state of a plurality of users in the district selected by the user on the map, and
wherein the communication unit is configured to transmit the map on which the average value is displayed to the terminal as information for indicating the average value of the severity of the mental state of the plurality of users.

10. The mental state classification server of claim 8, wherein the communication unit is configured to match the district selected by the user and the average value of the severity of the mental state of the plurality of users corresponding to the selected district and transmit them to the terminal.

11. The mental state classification server of claim 8,
wherein the processor is configured to: generate a plurality of subgroups by further classifying the plurality of groups for each job group; and calculate average values of severities of mental states of users of each of the plurality of subgroups, and
the communication unit is configured to transmit the average values of the severities of mental states of groups belonging to the job group selected by the user from among the plurality of subgroups to the terminal to display the average values.

12. The mental state classification server of claim 8,
wherein the processor is configured to: generate a plurality of age-subgroups by further classifying the plurality of groups by job group of the user and further classifying by age of the user, and control to display buttons indicating the age of each of the plurality of age-subgroups on the map,
wherein the communication unit is configured to receive an age selection signal about the age selected by the user’s terminal from the user’s terminal,
wherein the processor is configured to calculate the average value of the severity of the mental state of users of each of the plurality of age-subgroups, and
wherein the communication unit is configured to, in response to the received age selection signal, transmit the average values of the severities of mental states of a group belonging to the age selected by the terminal of the user among the plurality of age-subgroups to the terminal to display the average values.

13. The mental state classification server of claim 8,
wherein the communication unit is configured to receive comment contents input by the terminal of the user,
wherein the process is configured to: store the received comment contents in the memory, and register the received comment contents in a comment bulletin board, and
wherein the communication unit is configured to provide the comment bulletin board in which the comment contents are registered to a terminal of each of the plurality of users.

14. The mental state classification server of claim 8,
wherein the processor is configured to generate a mental state classification result report including a result of classifying all of the plurality of users and the mental state of at least one of groups in which the plurality of users is classified by department, and
wherein the communication unit is configured to transmit the generated mental state classification result report to a terminal of an administrator managing the plurality of users.
